SEQUENCE LISTING (69 sequences)

SEQ ID NO: 1 - protein, 4 aa, Artificial Sequence
  source: /note=“Description of Artificial Sequence Synthetic peptide”
RADA

SEQ ID NO: 2 - protein, 200 aa, Artificial Sequence
  source: /note=“Description of Artificial Sequence Synthetic polypeptide”
RADARADARADARADARADARADARADARADARADARADARADARADARADARADARADARADARADARADARADARADARADARADARADARADARADARADARADARADARADARADARADARADARADARADARADARADARADARADARADARADARADARADARADARADARADARADARADARADARADARADA

SEQ ID NO: 3 - protein, 4 aa, Artificial Sequence
  source: /note=“Description of Artificial Sequence Synthetic peptide”
IEIK

SEQ ID NO: 4 - protein, 201 aa, Artificial Sequence
  source: /note=“Description of Artificial Sequence Synthetic polypeptide”
IEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKI

SEQ ID NO: 5 - protein, 200 aa, Artificial Sequence
  source: /note=“Description of Artificial Sequence Synthetic polypeptide”
IEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIKIEIK

SEQ ID NO: 6 - protein, 4 aa, Artificial Sequence
  source: /note=“Description of Artificial Sequence Synthetic peptide”
KLDL

SEQ ID NO: 7 - protein, 200 aa, Artificial Sequence
  source: /note=“Description of Artificial Sequence Synthetic polypeptide”
KLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDLKLDL

SEQ ID NO: 8 - protein, 3 aa, Artificial Sequence
  source: /note=“Description of Artificial Sequence Synthetic peptide”
KLD

SEQ ID NO: 9 - protein, 150 aa, Artificial Sequence
  source: /note=“Description of Artificial Sequence Synthetic polypeptide”
KLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLDKLD

SEQ ID NO: 10 - protein, 16 aa, Artificial Sequence
  source: /note=“Description of Artificial Sequence Synthetic peptide”
RADARADARADARADA

SEQ ID NO: 11 - protein, 13 aa, Artificial Sequence
  source: /note=“Description of Artificial Sequence Synthetic peptide”
IEIKIEIKIEIKI

SEQ ID NO: 12 - protein, 17 aa, Artificial Sequence
  source: /note=“Description of Artificial Sequence Synthetic peptide”
IEIKIEIKIEIKIEIKI

SEQ ID NO: 13 - protein, 12 aa, Artificial Sequence
  source: /note=“Description of Artificial Sequence Synthetic peptide”
KLDLKLDLKLDL

SEQ ID NO: 14 - protein, 16 aa, Artificial Sequence
  source: /note=“Description of Artificial Sequence Synthetic peptide”
AEAEAKAKAEAEAKAK

SEQ ID NO: 15 - protein, 16 aa, Artificial Sequence
  source: /note=“Description of Artificial Sequence Synthetic peptide”
KAKAKAKAKAKAKAKA

SEQ ID NO: 16 - protein, 5 aa, Artificial Sequence
  source: /note=“Description of Artificial Sequence Synthetic peptide”
KAKAK

SEQ ID NO: 17 - protein, 16 aa, Artificial Sequence
  source: /note=“Description of Artificial Sequence Synthetic peptide”
AKAKAEAEAKAKAEAE

SEQ ID NO: 18 - protein, 16 aa, Artificial Sequence
  source: /note=“Description of Artificial Sequence Synthetic peptide”
AKAEAKAEAKAEAKAE

SEQ ID NO: 19 - protein, 16 aa, Artificial Sequence
  source: /note=“Description of Artificial Sequence Synthetic peptide”
EAKAEAKAEAKAEAKA

SEQ ID NO: 20 - protein, 8 aa, Artificial Sequence
  source: /note=“Description of Artificial Sequence Synthetic peptide”
AEAEAKAK

SEQ ID NO: 21 - protein, 12 aa, Artificial Sequence
  source: /note=“Description of Artificial Sequence Synthetic peptide”
AEAKAEAEAKAK

SEQ ID NO: 22 - protein, 16 aa, Artificial Sequence
  source: /note=“Description of Artificial Sequence Synthetic peptide”
KAEAKAEAKAEAKAEA

SEQ ID NO: 23 - protein, 16 aa, Artificial Sequence
  source: /note=“Description of Artificial Sequence Synthetic peptide”
AEAKAEAKAEAKAEAK

SEQ ID NO: 24 - protein, 8 aa, Artificial Sequence
  source: /note=“Description of Artificial Sequence Synthetic peptide”
ARARADAD

SEQ ID NO: 25 - protein, 16 aa, Artificial Sequence
  source: /note=“Description of Artificial Sequence Synthetic peptide”
ADADARARADADARAR

SEQ ID NO: 26 - protein, 16 aa, Artificial Sequence
  source: /note=“Description of Artificial Sequence Synthetic peptide”
ARADARADARADARAD

SEQ ID NO: 27 - protein, 16 aa, Artificial Sequence
  source: /note=“Description of Artificial Sequence Synthetic peptide”
DARADARADARADARA

SEQ ID NO: 28 - protein, 16 aa, Artificial Sequence
  source: /note=“Description of Artificial Sequence Synthetic peptide”
ADARADARADARADAR

SEQ ID NO: 29 - protein, 16 aa, Artificial Sequence
  source: /note=“Description of Artificial Sequence Synthetic peptide”
ARARADADARARADAD

SEQ ID NO: 30 - protein, 16 aa, Artificial Sequence
  source: /note=“Description of Artificial Sequence Synthetic peptide”
ARADAKAEARADAKAE

SEQ ID NO: 31 - protein, 16 aa, Artificial Sequence
  source: /note=“Description of Artificial Sequence Synthetic peptide”
AKAEARADAKAEARAD

SEQ ID NO: 32 - protein, 16 aa, Artificial Sequence
  source: /note=“Description of Artificial Sequence Synthetic peptide”
ARAKADAEARAKADAE

SEQ ID NO: 33 - protein, 16 aa, Artificial Sequence
  source: /note=“Description of Artificial Sequence Synthetic peptide”
AKARAEADAKARADAE

SEQ ID NO: 34 - protein, 16 aa, Artificial Sequence
  source: /note=“Description of Artificial Sequence Synthetic peptide”
AQAQAQAQAQAQAQAQ

SEQ ID NO: 35 - protein, 16 aa, Artificial Sequence
  source: /note=“Description of Artificial Sequence Synthetic peptide”
VQVQVQVQVQVQVQVQ

SEQ ID NO: 36 - protein, 16 aa, Artificial Sequence
  source: /note=“Description of Artificial Sequence Synthetic peptide”
YQYQYQYQYQYQYQYQ

SEQ ID NO: 37 - protein, 16 aa, Artificial Sequence
  source: /note=“Description of Artificial Sequence Synthetic peptide”
HQHQHQHQHQHQHQHQ

SEQ ID NO: 38 - protein, 16 aa, Artificial Sequence
  source: /note=“Description of Artificial Sequence Synthetic peptide”
ANANANANANANANAN

SEQ ID NO: 39 - protein, 16 aa, Artificial Sequence
  source: /note=“Description of Artificial Sequence Synthetic peptide”
VNVNVNVNVNVNVNVN

SEQ ID NO: 40 - protein, 16 aa, Artificial Sequence
  source: /note=“Description of Artificial Sequence Synthetic peptide”
YNYNYNYNYNYNYNYN

SEQ ID NO: 41 - protein, 16 aa, Artificial Sequence
  source: /note=“Description of Artificial Sequence Synthetic peptide”
HNHNHNHNHNHNHNHN

SEQ ID NO: 42 - protein, 16 aa, Artificial Sequence
  source: /note=“Description of Artificial Sequence Synthetic peptide”
ANAQANAQANAQANAQ

SEQ ID NO: 43 - protein, 16 aa, Artificial Sequence
  source: /note=“Description of Artificial Sequence Synthetic peptide”
AQANAQANAQANAQAN

SEQ ID NO: 44 - protein, 16 aa, Artificial Sequence
  source: /note=“Description of Artificial Sequence Synthetic peptide”
VNVQVNVQVNVQVNVQ

SEQ ID NO: 45 - protein, 16 aa, Artificial Sequence
  source: /note=“Description of Artificial Sequence Synthetic peptide”
VQVNVQVNVQVNVQVN

SEQ ID NO: 46 - protein, 16 aa, Artificial Sequence
  source: /note=“Description of Artificial Sequence Synthetic peptide”
YNYQYNYQYNYQYNYQ

SEQ ID NO: 47 - protein, 16 aa, Artificial Sequence
  source: /note=“Description of Artificial Sequence Synthetic peptide”
YQYNYQYNYQYNYQYN

SEQ ID NO: 48 - protein, 16 aa, Artificial Sequence
  source: /note=“Description of Artificial Sequence Synthetic peptide”
HNHQHNHQHNHQHNHQ

SEQ ID NO: 49 - protein, 16 aa, Artificial Sequence
  source: /note=“Description of Artificial Sequence Synthetic peptide”
HQHNHQHNHQHNHQHN

SEQ ID NO: 50 - protein, 18 aa, Artificial Sequence
  source: /note=“Description of Artificial Sequence Synthetic peptide”
AKAQADAKAQADAKAQAD

SEQ ID NO: 51 - protein, 18 aa, Artificial Sequence
  source: /note=“Description of Artificial Sequence Synthetic peptide”
VKVQVDVKVQVDVKVQVD

SEQ ID NO: 52 - protein, 18 aa, Artificial Sequence
  source: /note=“Description of Artificial Sequence Synthetic peptide”
YKYQYDYKYQYDYKYQYD

SEQ ID NO: 53 - protein, 18 aa, Artificial Sequence
  source: /note=“Description of Artificial Sequence Synthetic peptide”
HKHQHDHKHQHDHKHQHD

SEQ ID NO: 54 - protein, 4 aa, Artificial Sequence
  source: /note=“Description of Artificial Sequence Synthetic peptide”
ATAT

SEQ ID NO: 55 - protein, 4 aa, Artificial Sequence
  source: /note=“Description of Artificial Sequence Synthetic peptide”
TVTV

SEQ ID NO: 56 - protein, 4 aa, Artificial Sequence
  source: /note=“Description of Artificial Sequence Synthetic peptide”
ASAS

SEQ ID NO: 57 - protein, 4 aa, Artificial Sequence
  source: /note=“Description of Artificial Sequence Synthetic peptide”
SSSS

SEQ ID NO: 58 - protein, 7 aa, Artificial Sequence
  source: /note=“Description of Artificial Sequence Synthetic peptide”
VVVTTTT

SEQ ID NO: 59 - protein, 3 aa, Artificial Sequence
  source: /note=“Description of Artificial Sequence Synthetic peptide”
RAD

SEQ ID NO: 60 - protein, 7 aa, Artificial Sequence
  source: /note=“Description of Artificial Sequence Synthetic peptide”
AAAAAAK

SEQ ID NO: 61 - protein, 7 aa, Artificial Sequence
  source: /note=“Description of Artificial Sequence Synthetic peptide”
AAAAAAD

SEQ ID NO: 62 - protein, 8 aa, Artificial Sequence
  source: /note=“Description of Artificial Sequence Synthetic peptide”
ATATATAT

SEQ ID NO: 63 - protein, 8 aa, Artificial Sequence
  source: /note=“Description of Artificial Sequence Synthetic peptide”
TVTVTVTV

SEQ ID NO: 64 - protein, 8 aa, Artificial Sequence
  source: /note=“Description of Artificial Sequence Synthetic peptide”
ASASASAS

SEQ ID NO: 65 - protein, 7 aa, Artificial Sequence
  source: /note=“Description of Artificial Sequence Synthetic peptide”
SSSSSSS

SEQ ID NO: 66 - protein, 16 aa, Artificial Sequence
  source: /note=“Description of Artificial Sequence Synthetic peptide”
VRVRVDVDVRVRVDVD

SEQ ID NO: 67 - protein, 16 aa, Artificial Sequence
  source: /note=“Description of Artificial Sequence Synthetic peptide”
ADADAKAKADADAKAK

SEQ ID NO: 68 - protein, 16 aa, Artificial Sequence
  source: /note=“Description of Artificial Sequence Synthetic peptide”
EAEAEAEAEAEAEAEA

SEQ ID NO: 69 - protein, 16 aa, Artificial Sequence
  source: /note=“Description of Artificial Sequence Synthetic peptide”
ADADADADADADADAD

(19) United States
(12) Patent Application Publication
(10) Pub. No.: US 2016/0030628 A1
(43) Pub. Date: Feb. 4, 2016

(54) TREATMENT FOR BILE LEAKAGE

(71) Applicant: 3-D MATRIX, LTD, Tokyo (JP)

(72) Inventor: Satoru Kobayashi, Kanagawa (JP)

(73) Assignee: 3-D MATRIX, LTD., Tokyo (JP)

(21) Appl. No.: 14/776,140

(22) PCT Filed: Mar. 13, 2014
(86) PCT No.: PCT/IB2014/059765

§ 371 Date: Sep. 14, 2015

Related U.S. Application Data
(60) Provisional application No. 61/784,496, filed on Mar. 14, 2013.

(51) Int. Cl. A61L 24/10; A61L 24/00

(52) U.S. Cl. CPC A61L 24/10; A61L 24/0015; A61L 24/108; A61L 24/0031; A61L 24/001

(57) ABSTRACT

Materials and methods for treating bile leakage are disclosed. A peptide comprising between about 7 amino acids to about 32 amino acids may be introduced to a target site. The peptide may undergo self-assembly upon adjustment of a pH level of the solution to a physiological pH level.

### SEQUENCE LISTING

The instant application contains a Sequence Listing which has been submitted electronically in ASCII format and is hereby incorporated by reference in its entirety. Said ASCII copy, created Mar. 13, 2014, is named T2071-7013WO_SL and is 29,135 bytes in size.

### FIELD OF THE DISCLOSURE

This disclosure relates to materials and methods that may be used in medical, research, and industrial applications. More particularly, this disclosure relates to materials and methods that may be used to provide a treatment for bile leakage. The systems and methods to provide a treatment for bile leakage may prevent or reduce leakage of bile from a bile duct, gall bladder, liver, pancreas, duodenum, or duodenal papilla. The systems and methods may provide a physical barrier to reduce or prevent bile leakage.

### SUMMARY

In accordance with one or more aspects, a method of treating a bile leakage in a subject is provided. The method comprises positioning an end of a delivery device in a target area of the bile leakage in which an occlusion is desired. The method further comprises administering through the delivery device a solution comprising a self-assembling peptide comprising between about 7 amino acids and about 32 amino acids in an effective amount and in an effective concentration to form a hydrogel under conditions surrounding the bile leakage to provide an occlusion of the bile leakage. The method further comprises removing the delivery device from the target area of the bile leakage.

In accordance with one or more aspects, a kit for occluding a bile leakage in a subject is provided. The kit comprises a solution comprising a self-assembling peptide comprising between about 7 amino acids and about 32 amino acids in an effective amount and in an effective concentration to form a hydrogel under physiological conditions to provide occlusion of the bile leakage. The kit further comprises instructions for administering the solution to a target area of the bile leakage of the subject.

In accordance with one or more aspects, a macroscopic scaffold consisting essentially of a plurality of self-assembling peptides is provided. Each of the self-assembling peptides comprises between about 7 amino acids and about 32 amino acids in an effective amount that is capable of being positioned within a target area of a bile leakage to promote an occlusion and to prevent the bile leakage.

### DESCRIPTION OF THE FIGURES

FIG. 1A is an image of a needle puncturing a gall bladder, in accordance with some embodiments;

FIG. 1B is an image of a gall bladder with bile leakage, in accordance with some embodiments;

FIG. 1C is an image of a gall bladder with application of a peptide solution, in accordance with some embodiments;

FIG. 1D is an image of a gall bladder after application of a peptide solution, in accordance with some embodiments;

FIG. 1E is an image of a gall bladder after application of a peptide solution, in accordance with some embodiments; and

FIG. 2 is a histopathological image of a hematoxylin and eosin (H&E) stained specimen of a target area of bile leakage, in accordance with some embodiments.

### DETAILED DESCRIPTION

Materials and methods of the present disclosure may treat bile leakage.

Bile leakage is a condition that may occur in a subject. The bile leakage may occur post-operatively. It may present itself at any time after surgery, and may sometimes present itself within one week of surgery. It may also occur for up to one week or longer after surgery. Bile leakage may be a complication of a hepatectomy or a cholecystectomy. A hepactectomy refers to a resection or partial or total removal of the liver. A cholecystectomy refers to a surgical removal of a gall bladder. Bile leakage may also occur after endoscopic surgery or after hepato-biliary-pancreatic surgery. Bile leakage may also occur after a liver resection, a pancreatic resection, a pancreatoduodenectomy, or a cholecystectomy.

Bile leakage may occur from the common bile duct, which connects the cystic and common hepatic ducts to the duodenum. An injured bile duct may leak bile and may cause a painful and potentially dangerous infection. Many cases of minor injury to the bile duct may be managed non-surgically. Major injury to the bile duct may require corrective surgery.

The rate of incidence of bile leakage occurs in about 1 percent to about 2 percent of all surgeries. It generally does not cause death immediately, but decreases a subject's quality of life. If this complication does occur, a subject may have to wait for the injury to cure on its own, as there are no prophylaxes or treatments for these cases. The subject may have to use a drain.

If bile leakage doesn't stop, the patient cannot be taken off the drain and the period of hospitalization may be extended. For example, the period of hospitalization may be extended from about 1 week to about 2 months. In some instances, it may cause peritonitis or inflammation of the bowel tissue.

Presently, there are no approved materials for bile leakage treatment.

The present disclosure provides for a treatment for bile leakage. The treatment may comprise a self-assembling peptide hydrogel that may be used for bile leakage treatment. The bile leakage may present itself postoperatively. The treatment may include applying a peptide solution, peptide composition, membrane, hydrogel, or scaffold to a target area. The treatment may provide a physical barrier to prevent or reduce the bile leakage. The bile leakage may occur in one or more of a bile duct, gall bladder, liver, pancreas, duodenum, or duodenal papilla.

Preventing or reducing bile leakage may include providing an at least partial occlusion on at least partial obstruction of a bile leakage area. The bile leakage area may comprise a tear, cut, puncture, wound, or the like.

The materials and methods may comprise treatment of bile leakage in a subject. As used herein, the term “subject” is intended to include human and non-human animals, for example, vertebrates, large animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the invention includes all vertebrates, for example, non-mammals (such as birds, for example, chickens; amphibians; reptiles) and mammals, such as non-human primates, domesticated, and agriculturally useful animals, for example, sheep, dog, cat, cow, pig, rat, among others.

The materials and methods may include administration, application, or injection of a self-assembling peptide, or a solution comprising a self-assembling peptide, or a composition comprising a self-assembling peptide, to a predetermined or desired target area. The self-assembling peptide may be applied or introduced to the pre-determined or desired target area in the form of a self-assembling peptide solution, hydrogel, membrane, scaffold or other form. The pre-determined or desired target area may be at or near the location of a bile leakage, or other tear, cut, puncture, wound, or the like in the bile duct, gall bladder, liver, pancreas, duodenum, or duodenal papilla. The pre-determined or desired target area may be established based on the site of a surgical procedure or an unintentional or intentional trauma.

The term “self-assembling peptide” may refer to a peptide that may exhibit a beta-sheet structure in aqueous solution in the presence of specific conditions to induce the beta-sheet structure. These specific conditions may include increasing the pH of a self-assembling peptide solution. The increase in pH may be an increase in pH to a physiological pH. The specific conditions may also include adding a cation, such as a monovalent cation, to a self-assembling peptide solution. The specific conditions may include conditions related to a bile leakage.

The self-assembling peptide may be an amphiphilic self-assembling peptide. By “amphiphilic” it is meant that the peptide comprises hydrophobic portions and hydrophilic portions. In some embodiments, an amphiphilic peptide may comprise, consist essentially of, or consist of alternating hydrophobic amino acids and hydrophilic amino acids. By “alternating,” it is meant to include a series of three or more amino acids that alternate between a hydrophobic amino acid and a hydrophilic amino acid, and it need not include each and every amino acid in the peptide sequence alternating between a hydrophobic and a hydrophilic amino acid.

The self-assembling peptide, also referred to herein as “peptide” may be administered to the pre-determined or desired target area in the form of a self-assembling peptide solution, hydrogel, membrane, scaffold or other form. The hydrogel may also be referred to as a membrane or scaffold throughout this disclosure. The pre-determined or desired target area may be at or near the location of a bile leakage. The pre-determined or desired target area may be established based on the site of a surgical procedure, or an unintentional or intentional trauma.

The self-assembling peptide solution may be an aqueous self-assembling peptide solution. The self-assembling peptide may be administered, applied, or injected in a solution that is substantially cell-free, or free of cells. In certain embodiments, the self-assembling peptide may be administered, applied, or injected in a solution that is cell-free or free of cells.

The self-assembling peptide may also be administered, applied, or injected in a solution that is substantially drug-free or free of drugs. In certain embodiments, the self-assembling peptide may be administered, applied, or injected in a solution that is drug-free or free of drugs. In certain other embodiments, the self-assembling peptide may be administered, applied, or injected in a solution that is substantially cell-free and substantially drug-free. In still further certain other embodiments, the self-assembling peptide may be administered, applied, or injected in a solution that is cell-free and drug free.

The self-assembling peptide solution may comprise, consist of, or consist essentially of the self-assembling peptide. The self-assembling peptide may be in a modified or unmodified form. By “modified,” it is meant that the self-assembling peptide may have one or more domains that comprise one or more amino acids that, when provided in solution by itself, it would not self-assemble. By “unmodified,” it is meant that the self-assembling peptide may not have any other domains other than those that provide for self-assembly of the peptide. That is, an unmodified peptide consists of alternating hydrophobic and hydrophilic amino acids that may self-assemble into a beta-sheet structure, macroscopic structure, such as a hydrogelor scaffold.

Administration of a solution may comprise, consist of, or consist essentially of administration of a solution comprising, consisting of, or consisting essentially of self-assembling peptide comprising, consisting of, or consisting essentially at least about 7 amino acids. Administration of a solution may comprise, consist of, or consist essentially of administration of a solution comprising, consisting of, or consisting essentially of self-assembling peptide comprising, consisting of, or consisting essentially between about 7 amino acids and about 32 amino acids. Administration of a solution may comprise, consist of, or consist essentially of administration of a solution comprising, consisting of, or consisting essentially of self-assembling peptide comprising, consisting of, or consisting essentially between about 7 amino acids and 17 amino acids. Other peptides that do not comprise, consist of, or consist essentially of at least about 7 amino acids may be contemplated by this disclosure.

By alternating, it is meant to include a series of three or more amino acids that alternate between a hydrophobic amino acid and a hydrophilic amino acid, and it need not include each and every amino acid in the peptide sequence alternating between a hydrophobic and a hydrophilic amino acid.

The materials and methods may comprise administering a self-assembling peptide to a predetermined or desired target. The peptide may be administered as a hydrogel or form a hydrogel upon administration. A hydrogel is a term that may refer to a colloidal gel that is dispersed in water. The hydrogel may also be referred to as a membrane or scaffold throughout this disclosure. The systems and methods may also comprise applying a self-assembling peptide to a predetermined or desired target as a solution such as an aqueous peptide solution.

The term “administering,” is intended to include, but is not limited to, applying, introducing or injecting the self-assembling peptide, in one or more of various forms including, but not limited to, by itself, by way of solution, such as an aqueous solution, or by way of a composition, hydrogel, or scaffold, with or without additional components.

The method may comprise introducing a delivery device at or near a predetermined or desired target area of a subject. The method may comprise introducing a delivery device comprising at least one of a syringe, pipette, catheter, tube, syringe catheter, or other needle-based device to the predetermined or desired target area of a subject. The self-assembling peptide may be administered by way of a syringe, pipette, catheter, tube, syringe catheter, or other needle-based device to the predetermined or desired target area of a subject. The gauge of the syringe needle may be selected to provide an adequate flow of a composition, a solution, a hydrogel, or a liquid from the syringe to the target area. This may be based in some embodiments on at least one of the amount of self-assembling peptide in a composition, peptide solution, or a hydrogel being administered, the concentration of the peptide in solution, in the composition, or the hydrogel, and the viscosity of the peptide solution, composition, or hydrogel. The delivery device may be a conventional device or designed to accomplish at least one of to reach a specific target area, achieve a specific dosing regime, deliver a specific target volume, amount, or concentration, and delivery accurately to a target area.

The method of treating a bile leakage may comprise positioning an end of a delivery device in a predetermined or target area, such as a portion of a bile duct, gall bladder, liver, pancreas, duodenum, or duodenal papilla. The self-assembling peptide may be administered by way of a delivery device to the target area in which at least a partial occlusion of the bile leakage is desired. The use of a delivery device may provide a more selective administration of the peptide to provide for a more accurate delivery to the target area. Selective administration of the peptide may allow for enhanced and more targeted delivery of the peptide solution, composition, or hydrogel such that bile leakage occlusion is successful and positioned in the desired location in an accurate manner. The selective administration may provide enhanced, targeted delivery that markedly improves the positioning and effectiveness of the treatment over use of other delivery devices. Delivery devices that may be used in the systems, methods, and kits of the disclosure may include a syringe, needle, pipette, tube, syringe catheter, other needle-based device, or catheter.

Use of a catheter or syringe may include use of accompanying devices, such as a guidewire used to guide the catheter or syringe into position, or an endoscope that may allow for visualization of the target area and proper placement of the catheter. The endoscope may be a tube that may comprise at least one of a light and a camera or other visualization device to allow images of the subject's body to be viewed. The endoscope may be introduced to the subject prior to introducing the catheter or syringe to the subject.

The use of the delivery device, such as a syringe, needle, pipette, tube, syringe catheter, other needle-based device, catheter, or endoscope may require determining the diameter or size of the opening in which the device would be positioned at or near the target area, such that at least a portion of the delivery device may enter the opening to administer the peptide, peptide solution, hydrogel, or scaffold to the target area.

In certain embodiments, the hydrogel may be formed in vitro and administered to the desired location in vivo. In certain examples, this location may be the area in which it is desired to provide occlusion of bile leakage. In other examples, this location may be upstream of the area, downstream of the area, or substantially near the area. It may be desired to allow a migration of the hydrogel to the area in which it is desired to provide an occlusion of bile leakage. Alternatively, another procedure may position the hydrogel in the area in which it is desired. The desired location or target area may be at least a portion of an area in which tissue was removed, for example, in or around areas in which a tear, cut, puncture, wound, or the like exists. The desired location or target area may be at or near a location of a surgical procedure. The desired location or target area may be at or near a location of a hepatectomy or a cholecystectomy, or other surgical procedure that may cause bile leakage.

In certain aspects of the disclosure, the hydrogel may be formed in vivo. A solution comprising the self-assembling peptide, such as an aqueous solution, may be inserted to an in vivo location or area of a subject to provide an occlusion or blockage at that location. In certain examples, the hydrogel may be formed in vivo at one location, and allowed to migrate to the area in which it is desired to provide an occlusion or blockage at that location. The peptides of the present disclosure may be in the form of a powder, a solution, a gel, or the like. Since the self-assembling peptide gels in response to changes in solution pH and salt concentration, it can be distributed as a liquid that gels upon contact with a subject during application or administration.

In certain environments, the peptide solution may be a weak hydrogel and, as a result, it may be administered by way of a delivery device as described herein.

In accordance with one or more embodiments, a macroscopic scaffold is provided. The macroscopic scaffold may comprise, consist essentially of, or consist of a plurality of self-assembling peptides, each of which comprises, consists essentially of, or consists of at least about 7 amino acids in an effective amount that is capable of being positioned at or near a target site of a bile leakage to promote occlusion and prevent bile leakage. The macroscopic scaffold may comprise, consist essentially of, or consist of a plurality of self-assembling peptides, each of which comprises, consists essentially of, or consists of between about 7 amino acids and about 32 amino acids in an effective amount that is capable of being positioned at or near a target site of a bile leakage to promote occlusion and prevent bile leakage. The macroscopic scaffold may comprise, consist essentially of, or consist of a plurality of self-assembling peptides, each of which comprises, consists essentially of, or consists of between about 7 amino acids and about 17 amino acids in an effective amount that is capable of being positioned at or near a target site of a bile leakage to promote occlusion and prevent bile leakage. In accordance with some embodiments, the self-assembling peptides may be amphiphilic, alternating between hydrophobic amino acids and hydrophilic amino acids.

In accordance with one or more embodiments, a subject may be evaluated to determine a need for bile leakage occlusion. Once the evaluation has been completed, a peptide solution to administer to the subject may be prepared. The effect of introducing the self-assembling or self-organizing peptide may last for at least one month, and more typically may last for several months. This may be due to the sustainability of the gel in the body of the subject. Accordingly it is contemplated that this treatment may require only infrequent administration or doses or one administration or dose.

In some embodiments, a biologically active agent may be used with the materials and methods of the present disclosure.

A biologically active agent may comprise a compound, including a peptide, DNA sequence, chemical compound, or inorganic or organic compound that may impart some activity, regulation, modulation, or adjustment of a condition or other activity in a subject or in a laboratory setting. The biologically active agent may interact with another component to provide such activity. The biologically active agent may be referred to as a drug in accordance with some embodiments herein. In certain embodiments, one or more biologically active agents may be gradually released to the outside of the peptide system. For example, the one or more biologically active agents may be gradually released from the hydrogel. Both in vitro and in vivo testing has demonstrated this gradual release of a biologically active agent. The biologically active agent may be added to the peptide solution prior to administering to a subject, or may be administered separately from the solution to the subject.

The one or more biologically active agents may be a drug.

The biologically active agent may be gradually released to the outside of the peptide system. For example, the one or more biologically active agents may be gradually released from the hydrogel. A gradual release of a biologically active agent may be achieved in vitro and in vivo. The biologically active agent may be added to the peptide solution prior to administering to a subject, or may be administered separately from the solution to the subject.

This disclosure relates to aqueous solutions, hydrogels, scaffolds, and membranes comprising self-assembling peptides, sometimes referred to as self-assembling oligopeptides. The peptides may be comprised of a peptide having about 6 to about 200 amino acid residues. The self-assembling peptides may exhibit a beta-sheet structure in aqueous solution in the presence of physiological pH and/or a cation, such as a monovalent cation, or other conditions applicable to the target area of a bile leakage. The peptides may be amphiphilic and alternate between a hydrophobic amino acid and a hydrophilic amino acid. In certain embodiments, the peptide may comprise a first portion that may be amphiphilic, alternating between a hydrophobic amino acid and a hydrophilic amino acid, and another portion or region that is not amphiphilic. The peptides may be generally stable in aqueous solutions and self-assemble into large, macroscopic structures, scaffolds, or matrices when exposed to physiological conditions, neutral pH, or physiological levels of salt. Once the hydrogel is formed it may not decompose or biodegrade after a period of time, or may decompose or biodegrade after a period of time. The rate of decomposition may be based at least in part on at least one of the amino acid sequence and conditions of its surroundings.

By macroscopic it is meant as having dimensions large enough to be visible under magnification of 10-fold or less. In preferred embodiments, a macroscopic structure is visible to the naked eye. A macroscopic structure may be transparent and may be two-dimensional, or three-dimensional. Typically each dimension is at least 10 μm, in size. In certain embodiments, at least two dimensions are at least 100 μm, or at least 1000 μm in size. Frequently at least two dimensions are at least 1-10 mm in size, 10-100 mm in size, or more.

In certain embodiments, the size of the filaments may be about 10 nanometers (nm) to about 20 nm. The interfilament distance may be about 50 nm to about 80 nm. “Physiological” conditions may occur in nature for a particular organism, cell system, or subject which may be in contrast to artificial laboratory conditions. The conditions may comprise one or more properties such as one or more particular properties or one or more ranges of properties. For example, the physiological conditions may include a temperature or range of temperatures, a pH or range of pH's, a pressure or range of pressures, and one or more concentrations of particular compounds, salts, and other components. For example, in some examples, the physiological conditions may include a temperature in a range of about 20 to about 40 degrees Celsius. In some examples, the atmospheric pressure may be about 1 atm. The pH may be in the range of a neutral pH. For example, the pH may be in a range of about 6 to about 8. The physiological conditions may include cations such as monovalent metal cations that may induce membrane or hydrogel formation. These may include sodium chloride (NaCl). The physiological conditions may also include a glucose concentration, a sucrose concentration, or other sugar concentration of between about 1 mM and about 20 mM.

In some embodiments, the self-assembling peptides may be peptides of between about 6 amino acids and about 200 amino acids. In certain embodiments, the self-assembling peptides may be peptides of at least about 7 amino acids. In certain embodiments, the self-assembling peptides may be peptides of between about 7 amino acids and about 32 amino acids. In certain further embodiments, the self-assembling peptides may be peptides of between about 7 amino acids to about 17 amino acids. In certain other examples, the self-assembling peptides may be peptides of at least about 8 amino acids, at least about 12 amino acids, or at least about 16 amino acids. The self-assembling peptides may comprise, consist essentially of, or consist of an amphiphilic peptide that alternates between a hydrophobic amino acid and a hydrophilic amino acid.

The peptides may also be complementary and structurally compatible. Complementary refers to the ability of the peptides to interact through ionized pairs and/or hydrogen bonds which form between their hydrophilic side-chains, and structurally compatible refers to the ability of complementary peptides to maintain a constant distance between their peptide backbones. Peptides having these properties participate in intermolecular interactions which result in the formation and stabilization of beta-sheets at the secondary structure level and interwoven filaments at the tertiary structure level. Both homogeneous and heterogeneous mixtures of peptides characterized by the above-mentioned properties may form stable macroscopic membranes, filaments, and hydrogels. Peptides which are self-complementary and self-compatible may form membranes, filaments, and hydrogels in a homogeneous mixture. Heterogeneous peptides, including those which cannot form membranes in homogeneous solutions, which are complementary and/or structurally compatible with each other, may also self-assemble into macroscopic membranes and filaments.

The membranes, filaments, and hydrogels may be non-cytotoxic. The hydrogels of the present disclosure may be digested and metabolized in a subject. The hydrogels may be biodegraded in 30 days or less. They have a simple composition, are permeable, and are easy and relatively inexpensive to produce in large quantities. The membranes, filaments, hydrogel, or scaffolds may also be produced and stored in a sterile condition. The optimal lengths for membrane formation may vary with at least one of the amino acid composition, solution conditions, and conditions at the target site.

In certain embodiments, a method of treating a bile leakage in a subject is provided. The method may comprise positioning an end of a delivery device in a target area of the bile leakage in which an occlusion is desired. The method may also comprise administering through the delivery device a solution comprising a self-assembling peptide comprising at least about 7 amino acids in an effective amount and in an effective concentration to form a hydrogel under conditions surrounding the bile leakage to provide an occlusion of the bile leakage, and removing the delivery device from the target area of the bile leakage. In certain embodiments, the method may also comprise administering through the delivery device a solution comprising a self-assembling peptide comprising between about 7 amino acids and about 32 amino acids in an effective amount and in an effective concentration to form a hydrogel under conditions surrounding the bile leakage to provide an occlusion of the bile leakage, and removing the delivery device from the target area of the bile leakage. In certain other embodiments, the method may also comprise administering through the delivery device a solution comprising a self-assembling peptide comprising between about 7 amino acids and about 17 amino acids in an effective amount and in an effective concentration to form a hydrogel under conditions surrounding the bile leakage to provide an occlusion of the bile leakage, and removing the delivery device from the target area of the bile leakage.

The method may further comprise visualizing a region comprising at least a portion of the target area surrounding the bile leakage. Visualizing the region may occur during at least one of identifying the target area of the bile leakage, positioning the end of the delivery device in the target area, administering the solution, removing the syringe, and monitoring the bile leakage after removing the syringe. The bile leakage may occur from at least one of a bile duct, a gall bladder, liver, pancreas, duodenum, or duodenal papilla.

The solution to be administered may consist essentially of, or consist of, a self-assembling peptide comprising, consisting essentially of, or consisting of at least about 7 amino acids. The solution to be administered may consist essentially of, or consist of, a self-assembling peptide comprising, consist essentially of, or consist of between about 7 amino acids and about 32 amino acids. The solution to be administered may consist essentially of, or consist of, a self-assembling peptide comprising, consist essentially of, or consist of between about 7 amino acids and about 17 amino acids. The peptide may be amphiphilic and at least a portion of the peptide may alternate between a hydrophobic amino acid and a hydrophilic amino acid.

The method of treating may comprise visualizing the region in a time period about one minute, three minutes, and/or one week subsequent the administration. The effective amount and the effective concentration may be based in part on a dimension of the target area of the bile leakage. The effective amount may be approximately 1 mL per 1 cm² of target area. The concentration effective to provide the occlusion of the bile leakage may comprise a concentration in a range of about 0.1 weight per volume percent to about 3 weight per volume percent. The amount effective to provide the occlusion of the bile leakage may comprise a volume in a range of about 0.1 mol to about 5 mL.

The method of treating may comprise monitoring the area at the target area or the area surrounding the target area. The method may be used after a surgical procedure. The surgical procedure may be one of hepatectomy and cholecystectomy.

In certain embodiments of the present disclosure a method of facilitating occlusion of a bile leakage in a subject is provided. The method of facilitating may comprise providing a solution comprising a self-assembling peptide comprising at least about 7 amino acids in an effective amount and in an effective concentration to form a hydrogel under physiological conditions to provide the occlusion of the bile leakage. The method of facilitating may comprise providing a solution comprising a self-assembling peptide comprising between about 7 amino acids and about 32 amino acids in an effective amount and in an effective concentration to form a hydrogel under physiological conditions to provide the occlusion of the bile leakage. The method of facilitating may comprise providing a solution comprising a self-assembling peptide comprising between about 7 amino acids and about 17 amino acids in an effective amount and in an effective concentration to form a hydrogel under physiological conditions to provide the occlusion of the bile leakage. The peptide may be amphiphilic and at least a portion of the peptide may alternate between a hydrophobic amino acid and a hydrophilic amino acid.

The method of facilitating may also comprise providing instructions for administering the solution to a target area of the bile leakage through introduction of the solution through a delivery device positioned in the target area of the bile leakage. The method may further comprise providing instructions to visualize a region comprising at least a portion of the target area of the bile leakage. The method may further comprise providing instructions to visualize the region during at least one of identifying the target area of the bile leakage, positioning an end of the delivery device in the target area, administering the solution, removing the delivery device from the target area of the bile leakage, and monitoring the region after removing the syringe. The bile leakage may occur from at least one of a bile duct, a gall bladder, a liver, a pancreas, a duodenum, or a duodenal papilla.

The method of facilitating may comprise providing instructions to visualize the region in a time period about one minute, three minutes, and/or one week subsequent the administration. Instructions may be provided to monitor the area at the target area or surrounding the target area. Instructions may be provided to use the methods of the present disclosure after a surgical procedure. The surgical procedure may be one of hepatectomy and cholecystectomy.

The method of facilitating may further comprise providing instructions to prepare at least one of the effective amount and the effective concentration based in part on a dimension of the target area of the bile leakage. The effective amount may be approximately 1 mL per 1 cm² of target area. The concentration effective to provide the occlusion of the bile leakage may comprise a concentration in a range of about 0.1 weight per volume percent to about 3 weight per volume percent. The amount effective to provide the occlusion of the bile leakage may comprise a volume in a range of about 0.1 mL to about 5 mL.

The methods of the present disclosure may comprise evaluating the subject to determine a need for bile leakage occlusion and preparing the solution. The methods of the present disclosure may comprise visualizing the region wherein visualizing the region provides for selective administration to the target area of the bile leakage.

In accordance with some embodiments, a kit for occluding a bile leakage in a subject may be provided. The kit may comprise a solution comprising a self-assembling peptide comprising at least about 7 amino acids in an effective amount and in an effective concentration to form a hydrogel under physiological conditions to provide occlusion of the bile leakage. The kit may comprise providing a solution comprising a self-assembling peptide comprising between about 7 amino acids and about 32 amino acids in an effective amount and in an effective concentration to form a hydrogel under physiological conditions to provide the occlusion of the bile leakage. The method of facilitating may comprise providing a solution comprising a self-assembling peptide comprising between about 7 amino acids and about 17 amino acids in an effective amount and in an effective concentration to form a hydrogel under physiological conditions to provide the occlusion of the bile leakage.

The kit may further comprise instructions for administering the solution to a target area of the bile leakage of the subject. The kit may further comprise a delivery device, such as a syringe or syringe catheter to introduce the solution to the target area of the bile leakage of the subject. The kit may comprise a sucrose solution. Instructions for diluting the solution to administer an effective concentration of the solution to the target area of the bile leakage of the subject may also be provided. The instructions may describe diluting the peptide solution with a diluant or solvent. The diluant or solvent may be water. The kit may further comprise instructions for determining the effective concentration of the solution to the target area of the bile leakage in the subject based on a dimension of the target area of the bile leakage.

Other components or ingredients may be included in the kit, in the same or different compositions or containers than the peptide, peptide solutions, or hydrogel. The one or more components that may include components that may provide for enhanced effectiveness of the self-assembling peptide or may provide another action, treatment, therapy, or otherwise interact with one or more components of the subject. For example, additional peptides comprising one or more biologically or physiologically active sequences or motifs may be included as one of the components along with the self-assembling peptide. Other components may include biologically active compounds such as a drug or other treatment that may provide some benefit to the subject. For example, a cancer treating drug or anticancer drug may be administered with the self-assembling peptide, or may be administered separately. The peptide, peptide solution, or hydrogel may comprise small molecular drugs to treat the subject or to prevent hemolysis, inflammation, and infection, as disclosed herein. A sugar solution such as a sucrose solution may be provided with the kit. The sucrose solution may be a 20% sucrose solution.

Other components which are disclosed herein may also be included in the kit.

In some embodiments, a component of the kit is stored in a sealed vial, for example, with a rubber or silicone closure (for example, a polybutadiene or polyisoprene closure). In some embodiments, a component of the kit is stored under inert conditions (for example, under nitrogen or another inert gas such as argon). In some embodiments, a component of the kit is stored under anhydrous conditions (for example, with a desiccant). In some embodiments, a component of the kit is stored in a light blocking container such as an amber vial.

As part of the kit or separate from a kit, syringes or pipettes may be pre-filled with a peptide, peptide solution, or hydrogel as disclosed herein. Methods to instruct a user to supply a self-assembling peptide solution to a syringe or pipette, with or without the use of other devices, and administering it to the target area through the syringe or pipette, with or without the use of other devices, is provided. Other devices may include, for example, a catheter with or without a guidewire.

The amino acids of the self-assembling or amphiphilic peptides may be selected from d-amino acids, l-amino acids, or combinations thereof. The hydrophobic amino acids may include Ala, Val, Ile, Met, Phe, Tyr, Trp, Ser, Thr and Gly. The hydrophilic amino acids may be basic amino acids, for example, Lys, Arg, His, Orn; acidic amino acids, for example, Glu, Asp; or amino acids which form hydrogen bonds, for example, Asn, Gln. Acidic and basic amino acids may be clustered on a peptide. The carboxyl and amino groups of the terminal residues may be protected or not protected. Membranes or hydrogels may be formed in a homogeneous mixture of self-complementary and self-compatible peptides or in a heterogeneous mixture of peptides which are complementary and structurally compatible to each other. Peptides fitting the above criteria may self-assemble into macroscopic membranes under suitable conditions, described herein.

The self-assembling peptides may be composed of about 6 to about 200 amino acid residues. In certain embodiments, about 8 to about 32 residues may be used in the self-assembling peptides, while in other embodiments self-assembling peptides may have about 7 to about 17 residues. The peptides may have a length of about 5 nm. The peptides of the present disclosure may include peptides having the repeating sequence of arginine, alanine, aspartic acid and alanine (Arg-Ala-Asp-Ala (RADA) (SEQ ID NO: 1)), and such peptide sequences may be represented by (RADA)ₚ, wherein p=2-50 (SEQ ID NO: 2).

Other peptide sequences may be represented by self-assembling peptides having the repeating sequence of isoleucine, glutamic acid, isoleucine and lysine (Ile-Glu-Ile-Lys (IEIK) (SEQ ID NO: 3)), and such peptide sequences are represented by (IEIK)ₚ, wherein p=2-50 (SEQ ID NO: 5). Other peptide sequences may be represented by self-assembling peptides having the repeating sequence of isoleucine, glutamic acid, isoleucine and lysine (Ile-Glu-Ile-Lys (IEIK) (SEQ ID NO: 3)), and such peptide sequences are represented by (IEIK)ₚI, wherein p=2-50 (SEQ ID NO: 4).

Other peptide sequences may be represented by self-assembling peptides having the repeating sequence of lysine, leucine, aspartic acid, and leucine (Lys-Leu-Asp-Leu (KLDL) (SEQ ID NO: 6)), and such peptide sequences are represented by (KLDL)ₚ, wherein p=2-50 (SEQ ID NO: 7). Other peptide sequences may be represented by self-assembling peptides having the repeating sequence of lysine, leucine, and aspartic acid (Lys-Leu-Asp (KLD) (SEQ ID NO: 8)), and such peptide sequences are represented by (KLD)ₚ, wherein p=2-50 (SEQ ID NO: 9).

As specific examples of self-assembling peptides there may be a self-assembling peptide RADA16 having the sequence Arg-Ala-Asp-Ala-Arg-Ala-Asp-Ala-Arg-Ala-Asp-Ala-Arg-Ala-Asp-Ala (RADA)₄ (SEQ ID NO: 10), a self-assembling peptide IEIK13 having the sequence Ile-Glu-Ile-Lys-Ile-Glu-Ile-Lys-Ile-Glu-Ile-Lys-Ile (IEIK)₃I (SEQ ID NO: 11), a self-assembling peptide IEIK17 having the sequence Ile-Glu-Ile-Lys-Ile-Glu-Ile-Lys- Ile-Glu-Ile-Lys-Ile-Glu-Ile-Lys-Ile (IEIK)₄I (SEQ ID NO: 12) or a self-assembling peptide KLDL12 having the sequence Lys-Leu-Asp-Leu-Lys-Leu-Asp-Leu-Lys-Leu-Asp-Leu (KLDL)₃ (SEQ ID NO: 13).

Each of the peptide sequences disclosed herein may provide for peptides comprising, consisting essentially of, and consisting of the amino acid sequences recited.

The present disclosure provides materials, methods, and kits for solutions, hydrogels, and scaffolds comprising, consisting essentially of, or consisting of the peptides recited herein.

A 1 weight per volume (w/v) percent aqueous (water) solution and a 2.5 w/v percent of (RADA)₄ (SEQ ID NO: 10) is available as the product PuraMatrix™ peptide hydrogel by 3-D Matrix Co., Ltd.

Certain peptides may contain sequences which are similar to the cell attachment ligand RGD (Arginine-Glycine-Aspartic acid). The suitability of these peptides for supporting in vitro cell growth was tested by introducing a variety of cultured primary and transformed cells to homopolymer sheets of Ala-Glu-Ala-Glu-Ala-Lys-Ala-Lys-Ala-Glu-Ala-Glu-Ala-Lys-Ala-Lys (AEAEAKAKAEAEAKAK (EAK16) (SEQ ID NO: 14), RAD 16 (SEQ ID NO: 26), RADA16 (SEQ ID NO: 10), and heteropolymers of RAD16 (SEQ ID NO: 26) and EAK16 (SEQ ID NO: 14). The RAD-based peptides may be of interest because the similarity of this sequence to RGD. The RAD sequence is a high affinity ligand present in the extracellular matrix protein tenascin and is recognized by integrin receptors. The EAK16 peptide (SEQ ID NO: 14) and other peptides disclosed herein were derived from a region of a yeast protein, zuotin.

The self-assembly of the peptides may be attributable to hydrogen bonding and hydrophobic bonding between the peptide molecules by the amino acids composing the peptides.

In accordance with some embodiments, a macroscopic scaffold may be provided. The macroscopic scaffold may comprise, consist essentially of, or consist of a plurality of self-assembling peptides. Each of the self-assembling peptides comprising at least about 7 amino acids in an effective amount that is capable of being positioned within a target area of a bile leakage to promote occlusion and to prevent the bile leakage. Each of the self-assembling peptides may comprise, consist essentially of, or consist of between about 7 amino acids and about 32 amino acids in an effective amount that is capable of being positioned within a target area of a bile leakage to promote occlusion and to prevent the bile leakage. Each of the self-assembling peptides may comprise, consist essentially of, or consist of between about 7 amino acids and about 17 amino acids in an effective amount that is capable of being positioned within a target area of a bile leakage to promote occlusion and to prevent the bile leakage. In accordance with some embodiments, the self-assembling peptides may be amphiphilic, alternating between hydrophobic amino acids and hydrophilic amino acids.

The self-assembling peptides of the present disclosure may provide scaffolds having a nanofiber diameter in a range of about 10 nm to about 20 nm and an average pore size in a range of about 5 nm to about 200 nm. Each peptide may have a length of about 5 nanometers.

In certain embodiments, at least one of the nanofiber diameter, the pore size, and the nanofiber density may be controlled by at least one of the concentration of peptide solution used and the amount of peptide solution used, such as the volume of peptide solution. As such, at least one of a specific concentration of peptide in solution and a specific amount of peptide solution to provide at least one of a desired nanofiber diameter, pore size, and density to adequately deliver and form an embolism upon administration to a biological vessel may be selected.

As used herein, an amount of a peptide, peptide solution or hydrogel effective to at least partially occlude a bile leakage, an “effective amount” or a “therapeutically effective amount” refers to an amount of the peptide, peptide solution or hydrogel, which is effective, upon single or multiple administration (application or injection) to a subject, in treating, or in curing, alleviating, relieving or improving a subject with a disorder beyond that expected in the absence of such treatment. This may include a particular concentration or range of concentrations of peptide in the peptide solution or hydrogel and additionally, or in the alternative, a particular volume or range of volumes of the peptide solution or hydrogel. The method of facilitating may comprise providing instructions to prepare at least one of the effective amount and the effective concentration.

The dosage, for example, volume or concentration, administered (for example, applied or injected) may vary depending upon the form of the peptide (for example, in a peptide solution, hydrogel, or in a dried form, such as a lyophilized form) and the route of administration utilized. The exact formulation, route of administration, volume, and concentration can be chosen in view of the subject's condition and in view of the particular target area or location that the peptide solution, hydrogel, or other form of peptide will be administered. Lower or higher doses than those recited herein may be used or required. Specific dosage and treatment regimens for any particular subject may depend upon a variety of factors, which may include the specific peptide or peptides employed, the dimension of the area that is being treated, the desired thickness of the resulting hydrogel that may be positioned in the desired target area, and the length of time of treatment. Other factors that may affect the specific dosage and treatment regimens include age, body weight, general health status, sex, time of administration, rate of degradation, the severity and course of the disease, condition or symptoms, and the judgment of the treating physician. In certain embodiments, the peptide solution may be administered in a single dose. In other embodiments, the peptide solution may be administered in more than one dose, or multiple doses. The peptide solution may be administered in at least two doses.

An effective amount and an effective concentration of the peptide solution may be selected to at least occlude a bile leakage. In some embodiments, at least one of the effective amount and the effective concentration may be based in part on a dimension or diameter of the target area. In other embodiments, at least one of the effective amount and the effective concentration is based in part on the flow rate of one or more fluids at or near the target area.

In yet other embodiments, at least one of the effective amount and the effective concentration may be based in part on at least one of a dimension or diameter of the target area, the flow rate of one or more fluids at or near the target area, and on a dimension or diameter of a tear, cut, puncture, wound, or the like.

The effective amount may be, as described herein, an amount that may provide for an at least partial occlusion of the bile leakage. Various properties of the bile, bile duct, gall bladder, liver, pancreas, duodenum, or duodenal papilla may contribute to the selection or determination of the effective amount including at least one of the dimension or diameter of the target area, the flow rate of one or more fluids at or near the target area, the pH at or near the target area, and the concentration of various salts at or near the target area. Additional properties that may determine the effective amount include various properties listed above, at various locations along a pathway in which the peptide solution is delivered.

The effective amount may include volumes of from about 0.1 milliliters (mL) to about 100 mL of a peptide solution. The effective amount may include volumes of from about 0.1 mL to about 10 mL of a peptide solution. In certain embodiments, the effective amount may be about 0.5 mL. In other embodiments, the effective amount may be about 1.0 mL. In yet other embodiments, the effective amount may be about 1.5 mL. In still yet other embodiments, the effective amount may be about 2.0 mL. In some other embodiments, the effective amount may be about 3.0 mL. In certain embodiments, the effective amount may be approximately 0.1 mL to about 5 mL per 1 cm² of target area. In certain embodiments, the effective amount may be approximately 1 mL per 1 cm² of target area. This effective amount may be used related to a concentration of peptide in solution, such as a 2.5 weight per volume percent of a peptide solution of the present disclosure.

In some embodiments, a more effective treatment of bile leakage may be achieved with a greater volume of peptide solution administered or a higher concentration of peptide in solution to be administered. This may allow a longer or thicker hydrogel to form within the target area, allowing a more secure position of the hydrogel in the target area. It is possible that if a high enough volume is not selected, the hydrogel may not be effective in providing an occlusion of bile leakage in the target area for the desired period of time.

The effective concentration may be, as described herein, an amount that may provide for a desired occlusion of a bile leakage. Various properties of the target area may contribute to the selection or determination of the effective concentration including at least one of a dimension or diameter of the target area, the flow rate of one or more fluids at or near the target area, and on a dimension or diameter of a tear, cut, puncture, wound, or the like.

The effective concentration may include peptide concentrations in the solution in a range of about 0.1 weight per volume (w/v) percent to about 10 w/v percent. The effective concentration may include peptide concentrations in the solution in a range of about 0.1 w/v percent to about 3.5 w/v percent. In certain embodiments, the effective concentration may be about 1 w/v percent. In other embodiments, the effective concentration may be about 2.5 w/v percent. In yet other embodiments, the effective concentration may be about 3.0 w/v percent.

In certain embodiments, a peptide solution having a higher concentration of peptide may provide for a more effective hydrogel that has the ability to stay in place and provide effective treatment of bile leakage. For purposes of delivering the peptide solution, higher concentrations of peptide solutions may become too viscous to allow for effective and selective administration of the solution. It is possible that if a high enough concentration is not selected, the hydrogel may not be effective in maintaining an occlusion of the bile leakage in the target area for the desired period of time.

The effective concentration may be selected to provide for a solution that may be administered by injection or other means using a particular diameter or gauge catheter or needle, or other delivery device.

Methods of the disclosure contemplate single as well as multiple administrations of a therapeutically effective amount of the peptides, compositions, peptide solutions, membranes, filaments, and hydrogels as described herein. Peptides as described herein may be administered at regular intervals, depending on the nature, severity and extent of the subject's condition. In some embodiments, a peptide, composition, peptide solution, membrane, filament, or hydrogel may be administered in a single administration. In some embodiments, a peptide, composition, peptide solution, hydrogel, or scaffold described herein is administered in multiple administrations. In some embodiments, a therapeutically effective amount of a peptide, composition, peptide solution, membrane, filament, hydrogel, or scaffold may be administered periodically at regular intervals. The regular intervals selected may be based on any one or more of the initial peptide concentration of the solution administered, the amount administered, and the degradation rate of the hydrogel formed. For example, after an initial administration, one or more follow-on administrations may occur after, for example, one minute, two minutes, three minutes, ten minutes, 20 minutes, 30 minutes, or one hour. In some embodiments, after initial administration, one or more follow-on administrations may occur after, for example, one week, two weeks, four weeks, six weeks, or eight weeks. The follow-on administration may comprise administration of a solution having the same concentration of peptide and volume as the initial administration, or may comprise administration of a solution of lesser or greater concentration of peptide and volume. The selection of the appropriate follow-on administration of peptide solution may be based on visualizing or imaging the target area and the area surrounding the target area and ascertaining the needs based on the condition of the subject. The pre-determined intervals may be the same for each follow-on administration, or they may be different. In some embodiments, a peptide, peptide solution, or hydrogel may be administered chronically at pre-determined intervals to maintain at least a partial bile leakage occlusion in a subject over the life of the subject. The pre-determined intervals may be the same for each follow-on administration, or they may be different. This may be dependent on whether the hydrogel formed from the previous administration is partially or totally disrupted or degraded. The follow-on administration may comprise administration of a solution having the same concentration of peptide and volume as the initial administration, or may comprise administration of a solution of lesser or greater concentration of peptide and volume. The selection of the appropriate follow-on administration of peptide solution may be based on imaging the target area and the area surrounding the target area and ascertaining the needs based on the condition of the subject.

The self-assembling peptides of the present disclosure, such as RADA16 (SEQ ID NO: 10), may be peptide sequences that lack a distinct physiologically or biologically active motif or sequence, and therefore may not impair intrinsic cell function. Physiologically active motifs may control numerous intracellular phenomena such as transcription, and the presence of physiologically active motifs may lead to phosphorylation of intracytoplasmic or cell surface proteins by enzymes that recognize the motifs. When a physiologically active motif is present in a peptide tissue occluding agent, transcription of proteins with various functions may be activated or suppressed. The self-assembling peptides, of the present disclosure may lack such physiologically active motifs and therefore do not carry this risk.

A sugar may be added to the self-assembling peptide solution to improve the osmotic pressure of the solution from hypotonicity to isotonicity without reducing the tissue occluding effect, thereby allowing the biological safety to be increased. In certain examples, the sugar may be sucrose or glucose.

The optimal lengths for membrane formation may vary with the amino acid composition and conditions of the target area.

A stabilization factor contemplated by the peptides of the present disclosure is that complementary peptides maintain a constant distance between the peptide backbones. Peptides which can maintain a constant distance upon pairing are referred to herein as structurally compatible. The interpeptide distance can be calculated for each ionized or hydrogen bonding pair by taking the sum of the number of unbranched atoms on the side-chains of each amino acid in the pair. For example, lysine has 5 and glutamic acid has 4 unbranched atoms on its side-chains, respectively.

Other examples of peptides that may form membranes, hydrogels or scaffolds in homogeneous or heterogeneous mixtures are listed in Table 1.

TABLE 1
Potential membrane-forming peptides

| Name | Sequence (N→C) |
|---|---|
| KAKA16 | KAKAKAKAKAKAKAKA (SEQ ID NO: 15) |
| KAKA5 | KAKAK (SEQ ID NO: 16) |
| KAE16 | AKAKAEAEAKAKAEAE (SEQ ID NO: 17) |
| AKE16 | AKAEAKAEAKAEAKAE (SEQ ID NO: 18) |
| EKA16 | EAKAEAKAEAKAEAKA (SEQ ID NO: 19) |
| EAK8 | AEAEAKAK (SEQ ID NO: 20) |
| EAK12 | AEAKAEAEAKAK (SEQ ID NO: 21) |
| KEA16 | KAEAKAEAKAEAKAEA (SEQ ID NO: 22) |
| AEK16 | AEAKAEAKAEAKAEAK (SEQ ID NO: 23) |
| ARD8 | ARARADAD (SEQ ID NO: 24) |
| DAR16 | ADADARARADADARAR (SEQ ID NO: 25) |
| RAD16 | ARADARADARADARAD (SEQ ID NO: 26) |
| DRA16 | DARADARADARADARA (SEQ ID NO: 27) |
| RADA16 | RADARADARADARADA (SEQ ID NO: 10) |
| ADR16 | ADARADARADARADAR (SEQ ID NO: 28) |
| ARA16 | ARARADADARARADAD (SEQ ID NO: 29) |
| ARDAKE16 | ARADAKAEARADAKAE (SEQ ID NO: 30) |
| AKEW16 | AKAEARADAKAEARAD (SEQ ID NO: 31) |
| ARKADE16 | ARAKADAEARAKADAE (SEQ ID NO: 32) |
| AKRAED16 | AKARAEADAKARADAE (SEQ ID NO: 33) |
| AQ16 | AQAQAQAQAQAQAQAQ (SEQ ID NO: 34) |
| VQ16 | VQVQVQVQVQVQVQVQ (SEQ ID NO: 35) |
| YQ16 | YQYQYQYQYQYQYQYQ (SEQ ID NO: 36) |
| HQ16 | HQHQHQHQHQHQHQHQ (SEQ ID NO: 37) |
| AN16 | ANANANANANANANAN (SEQ ID NO: 38) |
| VN16 | VNVNVNVNVNVNVNVN (SEQ ID NO: 39) |
| YN16 | YNYNYNYNYNYNYNYN (SEQ ID NO: 40) |
| HN16 | HNHNHNHNHNHNHNHN (SEQ ID NO: 41) |
| ANQ16 | ANAQANAQANAQANAQ (SEQ ID NO: 42) |
| AQN16 | AQANAQANAQANAQAN (SEQ ID NO: 43) |
| VNQ16 | VNVQVNVQVNVQVNVQ (SEQ ID NO: 44) |
| VQK16 | VQVNVQVNVQVNVQVN (SEQ ID NO: 45) |
| YNQ16 | YNYQYNYQYNYQYNYQ (SEQ ID NO: 46) |
| YQN16 | YQYNYQYNYQYNYQYN (SEQ ID NO: 47) |
| HNQ16 | HNHQHNHQHNHQHNHQ (SEQ ID NO: 48) |
| HQN16 | HQHNHQHNHQHNHQHN (SEQ ID NO: 49) |
| AKQD18 | AKAQADAKAQADAKAQAD (SEQ ID NO: 50) |
| VKQ18 | VKVQVDVKVQVDVKVQVD (SEQ ID NO: 51) |
| YKQ18 | YKYQYDYKYQYDYKYQYD (SEQ ID NO: 52) |
| HKQ18 | HKHQHDHKHQHDHKHQHD (SEQ ID NO: 53) |
| | RADA (SEQ ID NO: 1) |
| | IEIK (SEQ ID NO: 3) |
| | ATAT (SEQ ID NO: 54) |
| | TVTV (SEQ ID NO: 55) |
| | ASAS (SEQ ID NO: 56) |
| | SSSS (SEQ ID NO: 57) |
| | VVVTTTT (SEQ ID NO: 58) |
| | RAD (SEQ ID NO: 59) |
| | KLD (SEQ ID NO: 8) |
| | AAAAAAK (SEQ ID NO: 60) |
| | AAAAAAD (SEQ ID NO: 61) |
| | ATATATAT (SEQ ID NO: 62) |
| | TVTVTVTV (SEQ ID NO: 63) |
| | ASASASAS (SEQ ID NO: 64) |
| | SSSSSSS (SEQ ID NO: 65) |

The criteria of amphiphilic sequence, length, complementarity and structural compatibility apply to heterogeneous mixtures of peptides. For example, two different peptides may be used to form the membranes: peptide A, Val-Arg-Val-Arg-Val-Asp-Val-Asp-Val-Arg-Val-Arg-Val-Asp-Val-Asp (VRVRVDVDVRVRVDVD (SEQ ID NO: 66), has Arg and Asp as the hydrophilic residues and peptide B, ADADAKAKADADAKAK (SEQ ID NO: 67), has Lys and Asp. Peptides A and B are complementary; the Arg on A can form an ionized pair with the Asp on B and the Asp on A can form an ionized pair with the Lys on B. Thus, in a heterogeneous mixture of peptides A and B, membranes would likely form, but they would be homogeneously composed of either peptide A or B.

Membranes and hydrogels can also be formed of heterogeneous mixtures of peptides, each of which alone would not form membranes, if they are complementary and structurally compatible to each other. For example, mixtures of (Lys-Ala-Lys-Ala)₄ (KAKA)₄ (SEQ ID NO: 15) and (Glu-Ala-Glu-Ala)₄ (EAEA)₄ (SEQ ID NO: 68) or of (Lys-Ala-Lys-Ala)₄ (KAKA)₄ (SEQ ID NO: 15) and (Ala-Asp-Ala-Asp)₄ (ADAD)₄ (SEQ ID NO: 69) would be expected to form membranes, but not any of these peptides alone due to lack of complementarity.

Peptides, which are not perfectly complementary or structurally compatible, can be thought of as containing mismatches analogous to mismatched base pairs in the hybridization of nucleic acids. Peptides containing mismatches can form membranes if the disruptive force of the mismatched pair is dominated by the overall stability of the interpeptide interaction. Functionally, such peptides can also be considered as complementary or structurally compatible. For example, a mismatched amino acid pair may be tolerated if it is surrounded by several perfectly matched pairs on each side. The peptides can be chemically synthesized or they can be purified from natural and recombinant sources. Using chemically synthesized peptides may allow the peptide solutions to be deficient in unidentified components such as unidentified components derived from the extracellular matrix of another animal. This property therefore may eliminate concerns of infection, including risk of viral infection compared to conventional tissue-derived biomaterials. This may eliminate concerns of infection including infections such as bovine spongiform encephalopathy (BSE), making the peptide highly safe for medical use.

The initial concentration of the peptide may be a factor in the size and thickness of the membrane, hydrogel, or scaffold formed. In general, the higher the peptide concentration, the higher the extent of membrane formation. Hydrogels, or scaffolds formed at higher initial peptide concentrations (about 10 mg/ml) (about 1.0 w/v percent) may be thicker and thus, likely to be stronger.

Formation of the membranes, hydrogels, or scaffolds may be very fast, on the order of a few minutes. In certain embodiments, the formation may be reversible, and in other embodiments, the formation may be irreversible

The hydrogel may form instantaneously upon administration to a desired or target area. The formation of the hydrogel may occur within about one to two minutes of administration. In other examples, the formation of the hydrogel may occur within about three to four minutes of administration. In certain embodiments, the time it takes to form the hydrogel may be based at least in part on one or more of the concentration of the peptide solution, the volume of peptide solution applied, and the conditions at the area of application or injection (for example, the concentration of monovalent metal cations at the area of application, the pH of the area, and the presence of one or more fluids at or near the area). In certain embodiments, the formation may be reversible, and in other embodiments, the formation may be irreversible. The process may be unaffected by pH of less than or equal to 12, and by temperature. The hydrogel may form at temperatures in the range of 1 to 99 degrees Celsius.

The hydrogels may remain in position at the target area for a period of time sufficient to provide a desired effect using the methods and kits of the present disclosure. The desired effect may be to at least partially occlude a bile leakage.

The desired effect using the methods and kits of the present disclosure may be to treat areas or to assist in healing of areas in which a surgical procedure in a subject is performed. The desired effect using the methods and kits of the present disclosure may be to treat areas or to assist in healing of areas in which a surgical procedure of the gall bladder, bile duct, or liver was performed. For example, the desired effect using the methods and kits of the present disclosure may be to treat areas or to assist in healing of areas in which bile leakage has occurred. This may include a surgical procedure of a hepatectomy or a cholecystectomy, in which a complication occurred during surgery to present the bile leakage.

The period of time that the membranes or hydrogels may remain at the desired area may be for about 10 minutes. In certain examples, it may remain at the desired area for about 35 minutes. In certain further examples, it may remain at the desired area for several days, up to two weeks. In other examples, it may remain at the desired area for up to 30 days, or more. It may remain at the desired area indefinitely. In other examples, it may remain at the desired area for a longer period of time, until it is naturally degraded or intentionally removed. If the hydrogel naturally degrades over a period of time, subsequent application or injection of the hydrogel to the same or different location may be performed.

In certain embodiments, the self-assembling peptide may be prepared with one or more components that may provide for enhanced effectiveness of the self-assembling peptide or may provide another action, treatment, therapy, or otherwise interact with one or more components of the subject. For example, additional peptides comprising one or more biologically or physiologically active amino acid sequences or motifs may be included as one of the components along with the self-assembling peptide. Other components may include biologically active compounds such as a drug or other treatment that may provide some benefit to the subject. For example, a cancer treating drug or anticancer drug may be administered with the self-assembling peptide, or may be administered separately.

The peptide, peptide solution, or hydrogel may comprise small molecular drugs to treat the subject or to prevent hemolysis, inflammation, and infection. The small molecular drugs may be selected from the group consisting of glucose, saccharose, purified saccharose, lactose, maltose, trehalose, destran, iodine, lysozyme chloride, dimethylisoprpylazulene, tretinoin tocoferil, povidone iodine, alprostadil alfadex, anise alcohol, isoamyl salicylate, α,α-dimethylphenylethyl alcohol, bacdanol, helional, sulfazin silver, bucladesine sodium, alprostadil alfadex, gentamycin sulfate, tetracycline hydrochloride, sodium fusidate, mupirocin calcium hydrate and isoamyl benzoate. Other small molecular drugs may be contemplated. Protein-based drugs may be included as a component to be administered, and may include erythropoietin, tissue type plasminogen activator, synthetic hemoglobin and insulin.

A component may be included to protect the peptide solution against rapid or immediate formation into a hydrogel. This may include an encapsulated delivery system that may degrade over time to allow a controlled time release of the peptide solution into the target area to form the hydrogel over a desired, predetermined period of time. Biodegradable, biocompatible polymers may be used, such as ethylene vinyl acetate, polyanhydrides, polyglycolic acid, collagen, polyorthoesters, and polylactic acid.

Any of the components described herein may be included in the peptide solution or may be administered separate from the peptide solution. Additionally, any of the methods and methods of facilitating provided herein may be performed by one or more parties.

Modification of the membranes may give them additional properties. For example, the membranes may be further strengthened by cross-linking the peptides after membrane formation by standard methods. Collagen may be combined with the peptides to produce membranes more suitable for use as artificial skin; the collagen may be stabilized from proteolytic digestion within the membrane. Furthermore, combining phospholipids with the peptides may produce vesicles.

In some embodiments of the disclosure, the self-assembling peptides may be used as a coating on a device or an instrument such as a stent or catheter, to suppress body fluid leakage. The self-assembling peptides may also be incorporated or secured to a support, such as gauze or a bandage, or a lining, that may provide a therapeutic effect to a subject, or that may be applied within a target area. The self-assembling peptides may also be soaked into a sponge for use.

The membranes may also be useful for culturing cell monolayers. Cells prefer to adhere to non-uniform, charged surfaces. The charged residues and conformation of the proteinaceous membranes promote cell adhesion and migration. The addition of growth factors, such as fibroblast growth factor, to the peptide membrane may further improve attachment, cell growth and neurite outgrowth.

### EXAMPLE

The objective of this study was to evaluate a self-assembling peptide solution (RADA16 in the form of PuraMatrix™ peptide hydrogel by 3-D Matrix, LTD.) as a bile leakage occlusion material in a porcine model. A histopathology assessment was also performed.

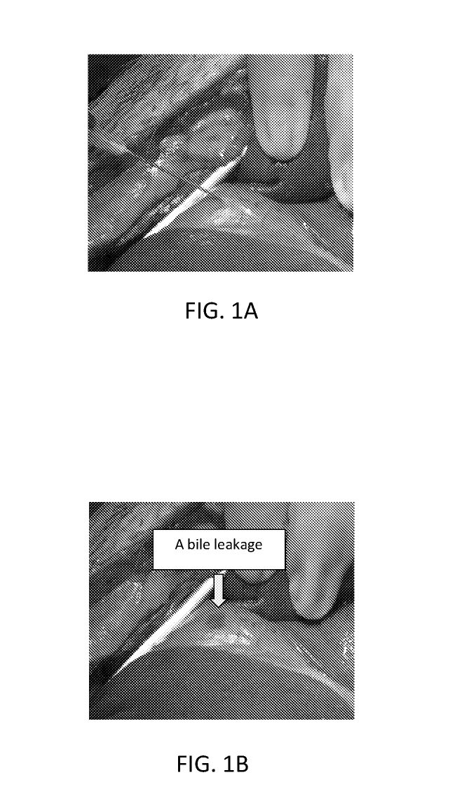

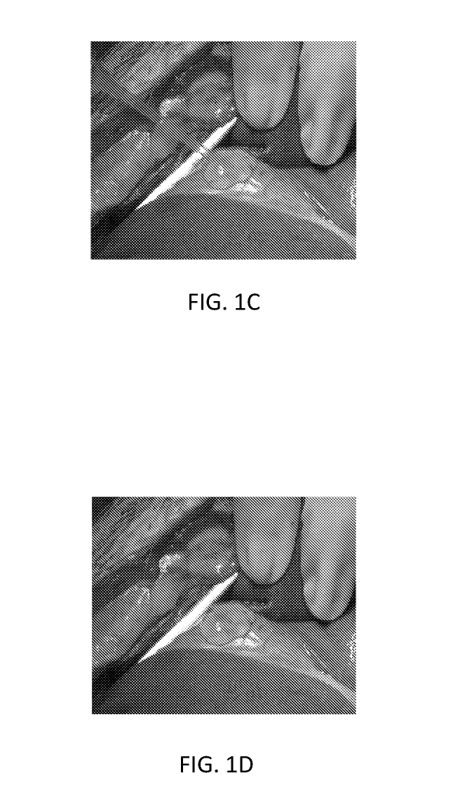

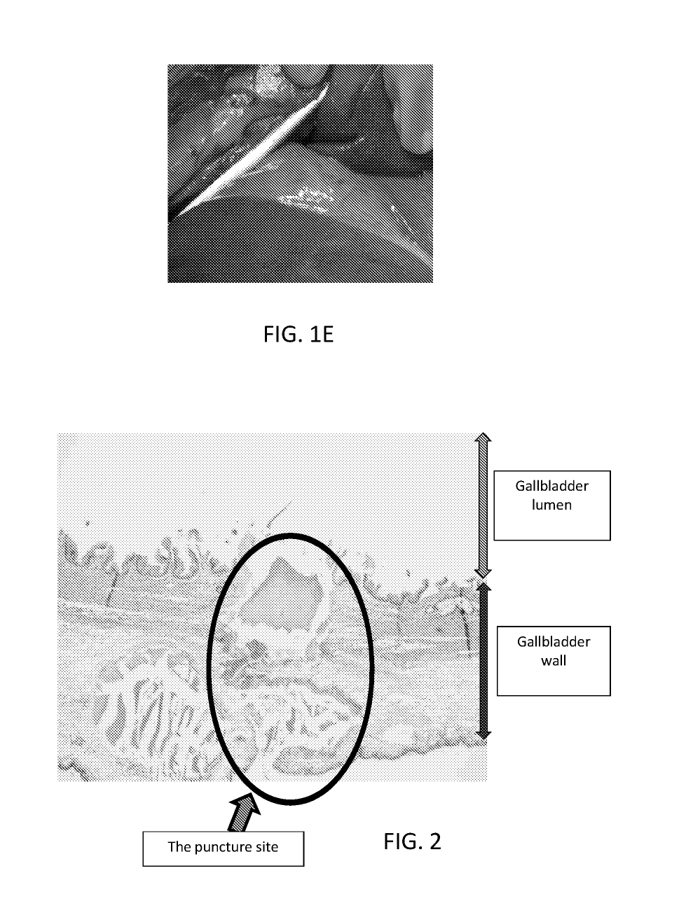

On the day of testing, an animal was sedated and prepared for surgery. A bile leakage model was prepared by puncturing needle punctures, using a 20G injection needle in the gall bladder of the test subject, as shown in FIG. 1A.

After a bile leakage was confirmed as shown in FIG. 1B, a 2.5% weight per volume percent of RADA16 was administered to the bile leakage point as shown in FIG. 1C to form a hydrogel with a syringe catheter. Subsequent to applying the self-assembling peptide solution, a bile leakage occlusion was confirmed at 1 minute, 20 seconds, as shown in FIG. 1D. Excess hydrogel was irrigated from the site and absence of a secondary bile leakage was confirmed at 3 minutes, as shown in FIG. 1E.

It was determined that 0.5 mL of the peptide solution was used to achieve the bile leakage occlusion of the needle puncture in the gallbladder.

After bile leakage occlusion was achieved, the gallbladder was fixed in formalin and the H&E-stained pathological specimen was made at the bile leakage occlusion site. As shown in FIG. 2, the bile leakage occlusion site occluded the bile leakage site.

The Example demonstrates the effectiveness and efficiency, as well as the rapidness of treatment of bile leakage in a subject. The materials and methods of the present disclosure have the ability to provide successful occlusion of bile leakage at a target area.

1. A method of treating a bile leakage in a subject comprising:
positioning an end of a delivery device in a target area of the bile leakage in which an occlusion is desired;
administering through the delivery device a solution comprising a self-assembling peptide comprising between about 7 amino acids and about 32 amino acids in an effective amount and in an effective concentration to form a hydrogel under conditions surrounding the bile leakage to provide an occlusion of the bile leakage;
removing the delivery device from the target area of the bile leakage.

2. The method of claim 1, further comprising visualizing a region comprising at least a portion of the target area surrounding the bile leakage.

3. The method of claim 2, wherein visualizing the region comprises visualizing the region during at least one of:
identifying the target area of the bile leakage;
positioning the end of the delivery device in the target area;
administering the solution;
removing the delivery device; and
monitoring the bile leakage after removing the delivery device.

4. The method of claim 3, wherein visualizing the region provides for selective administration of the solution to the target area of the bile leakage.

5. The method of claim 3, further comprising visualizing the region in a time period of about one minute subsequent to administering the solution.

6. The method of claim 5, further comprising visualizing the region in a time period of about three minutes subsequent to administering the solution.

7. The method of claim 6, further comprising visualizing the region in a time period of about one week subsequent to administering the solution.

8. The method of claim 1, wherein at least one of the effective amount and the effective concentration is based in part on a dimension of the target area of the bile leakage.

9. The method of claim 1, wherein the effective amount is approximately 1 mL per 1 cm² of target area.

10. The method of claim 1, wherein the concentration effective to provide the bile leakage occlusion comprises a concentration in a range of about 0.1 weight per volume (w/v) percent to about 3 w/v percent peptide.

11. The method of claim 1, wherein the amount effective to provide the bile leakage occlusion comprises a volume in a range of about 0.1 mL to about 5 mL.

12. The method of claim 1, further comprising monitoring the target area to determine an effectiveness of the administration of the solution.

13. The method of claim 1, further comprising performing a surgical procedure prior to positioning the delivery device in the target area.

14. The method of claim 13, wherein the surgical procedure is one of hepatectomy and cholecystectomy.

15. The method of claim 1, wherein the solution is substantially free of cells.

16. The method of claim 1, wherein the solution is substantially free of drugs.

17. The method of claim 1, wherein the solution consists essentially of a self-assembling peptide comprising between about 7 amino acids and about 32 amino acids.

18. The method of claim 17, wherein the solution consists of a self-assembling peptide comprising between about 7 amino acids and about 32 amino acids.

19. The method of claim 1, wherein the subject is a mammal.

20. The method of claim 19, wherein the subject is human.

21. The method of claim 1, wherein administering the solution comprises administering the solution in a single dose.

22. The method of claim 1, wherein administering the solution comprises administering the solution in at least two doses.

23. The method of claim 1, further comprising evaluating the subject to determine a need for preventing bile leakage occlusion and preparing the solution.

24. The method of claim 1, wherein the bile leakage is in at least one of a bile duct, a gall bladder, and a liver.

25. The method of claim 1, wherein the solution further comprises at least one biologically active agent.

26. The method of claim 1, wherein the peptide in the solution comprises one of (RADA)ₚ, wherein p=2-50 (SEQ ID NO: 2), (IEIK)ₚI, wherein p=2-50 (SEQ ID NO: 4), and (KLDL)ₚ, wherein p=2-50 (SEQ ID NO: 7).

27. The method of claim 26, wherein the peptide in the solution consists essentially of one of (RADA)ₚ, wherein p=2-50 (SEQ ID NO: 2), (IEIK)ₚI, wherein p=2-50 (SEQ ID NO: 4)), and (KLDL)ₚ, wherein p=2-50 (SEQ ID NO: 7).

28. The method of claim 1, wherein the peptide in the solution comprises one of (RADA)₄ (SEQ ID NO: 10), (IEIK)₃I (SEQ ID NO: 11), and (KLDL)₃ (SEQ ID NO: 13).

29. The method of claim 28, wherein the peptide in the solution consists essentially of (RADA)₄ (SEQ ID NO: 10), (IEIK)₃I (SEQ ID NO: 11), and (KLDL)₃ (SEQ ID NO: 13).

30. A kit for occluding a bile leakage in a subject comprising:
a solution comprising a self-assembling peptide comprising between about 7 amino acids and about 32 amino acids in an effective amount and in an effective concentration to form a hydrogel under physiological conditions to provide occlusion of the bile leakage; and
instructions for administering the solution to a target area of the bile leakage of the subject.

31. The kit of claim 30, further comprising a delivery device to introduce the solution to the target area of the bile leakage of the subject.

32. The kit of claim 30, further comprising a sucrose solution.

33. The kit of claim 30, further comprising instructions for diluting the solution to administer an effective concentration of the solution to the target area of the bile leakage of the subject.

34. The kit of claim 30, further comprising instructions for determining the effective concentration of the solution to the target area of the bile leakage in the subject based on a dimension of the target area of the bile leakage.

35. A method of facilitating occlusion of a bile leakage in a subject comprising:
providing a solution comprising a self-assembling peptide comprising between about 7 amino acids and about 32 amino acids in an effective amount and in an effective concentration to form a hydrogel under physiological conditions to provide occlusion of the bile leakage; and
providing instructions for administering the solution to a target area of the bile leakage through introduction of the solution through a delivery device positioned in the target area of the bile leakage.

36. The method of claim 35, further comprising providing instructions to visualize a region comprising at least a portion of the target area of the bile leakage.

37. The method of claim 36, wherein providing instructions to visualize the region comprising at least a portion of the target area of the bile leakage comprises providing instructions to visualize the region during at least one of:
identifying the target area of the bile leakage;
positioning an end of the delivery device in the target area;
administering the solution;
removing the delivery device from the target area of the bile leakage; and
monitoring the region after removing the delivery device.

38. The method of claim 36, wherein the bile leakage is in at least one of a bile duct, a gall bladder, and a liver.

39. The method of claim 36, further comprising providing instructions to visualize the region in a time period of about one minute subsequent to administering the solution.

40. The method of claim 39, further comprising providing instructions to visualize the region in a time period of about three minutes subsequent to administering the solution.

41. The method of claim 38, further comprising providing instructions to visualize the region in a time period of about one week subsequent to administering the solution.

42. The method of claim 35, further comprising providing instructions to prepare at least one of the effective amount and the effective concentration based in part on a dimension of the target area of the bile leakage.

43. The method of claim 35, wherein the effective amount is approximately 1 mL per 1 cm² of target area.

44. The method of claim 35, wherein the concentration effective to provide the occlusion of the bile leakage comprises a concentration in a range of about 0.1 weight per volume percent to about 3 weight per volume percent peptide.

45. The method of claim 35, wherein the amount effective to provide the occlusion of the bile leakage comprises a volume in a range of about 0.1 mL to about 5 mL.

46. The method of claim 35, further comprising providing instructions to monitor the area surrounding the target area.

47. The method of claim 35, further comprising providing the solution and instructions for use after a surgical procedure.

48. The method of claim 47, wherein the surgical procedure is one of hepatectomy and cholecystectomy.

49. The method of claim 35, wherein the solution is substantially free of cells.

50. The method of claim 35, wherein the solution is substantially free of drugs.

51. The method of claim 35, wherein the solution consists essentially of a self-assembling peptide comprising between about 7 amino acids and about 32 amino acids.

52. The method of claim 51, wherein the solution consists of a self-assembling peptide comprising between about 7 amino acids and about 32 amino acids.

53. The method of claim 35, wherein the subject is a mammal.

54. The method of claim 53, wherein the subject is human.

55. The method of claim 35, wherein administering the solution comprises administering the solution in a single dose.

56. The method of claim 35, wherein administering the solution comprises administering the solution in at least two doses.

57. The method of claim 35, further comprising evaluating the subject to determine a need for bile leakage occlusion and preparing the solution.

58. The method of claim 35, wherein visualizing the region provides for selective administration of the solution to the target area of the bile leakage.

59. The method of claim 35, wherein the solution further comprises at least one biologically active agent.

60. The method of claim 35, wherein the peptide in the solution comprises one of (RADA)ₚ, wherein p=2-50 (SEQ ID NO: 2), (IEIK)ₚI, wherein p=2-50 (SEQ ID NO: 4), and (KLDL)ₚ, wherein p=2-50 (SEQ ID NO: 7).

61. The method of claim 60, wherein the peptide in the solution consists essentially of one of (RADA)ₚ, wherein p=2-50 (SEQ ID NO: 2), (IEIK)ₚI, wherein p=2-50 (SEQ ID NO: 4), and (KLDL)ₚ, wherein p=2-50 (SEQ ID NO: 7).

62. The method of claim 35, wherein the peptide in the solution comprises one of (RADA)₄ (SEQ ID NO:10), (IEIK)₃I (SEQ ID NO: 11), and (KLDL)₃ (SEQ ID NO: 13).

63. The method of claim 62, wherein the peptide in the solution consists essentially of (RADA)₄ (SEQ ID NO: 10), (IEIK)₃I (SEQ ID NO: 11), and (KLDL)₃ (SEQ ID NO: 13).

64. A macroscopic scaffold consisting essentially of a plurality of self-assembling peptides, each of the self-assembling peptides comprising between about 7 amino acids and about 32 amino acids in an effective amount that is capable of being positioned within a target area of a bile leakage to promote an occlusion and to prevent the bile leakage.

65. The macroscopic scaffold of claim 64, wherein each of the plurality of peptides comprises one of (RADA)ₚ, wherein p=2-50 (SEQ ID NO: 2) and (IEIK)ₚI, wherein p=2-50 (SEQ ID NO: 4).

66. The macroscopic scaffold of claim 65, wherein each of the plurality of peptides consists essentially of one of (RADA)ₚ, wherein p=2-50 (SEQ ID NO: 2), (IEIK)ₚI, wherein p=2-50 (SEQ ID NO: 4), and (KLDL)ₚ, wherein p=2-50 (SEQ ID NO: 7).

67. The macroscopic scaffold of claim 64, wherein each of the plurality of peptides comprises one of (RADA)ₚ, wherein p=2-50 (SEQ ID NO: 2), (IEIK)ₚI, wherein p=2-50 (SEQ ID NO: 4), and (KLDL)ₚ, wherein p=2-50 (SEQ ID NO: 7).

68. The macroscopic scaffold of claim 67, wherein each of the plurality of peptides consists essentially of (RADA)₄ (SEQ ID NO: 10), (IEIK)₃I (SEQ ID NO: 11), and (KLDL)₃ (SEQ ID NO: 13).

69. The macroscopic scaffold of claim 65, comprising nanofibers having a diameter of about 10 nanometers to about 20 nanometers.

70. The macroscopic scaffold of claim 69, comprising nanofibers having a pore size of about 5 nanometers to about 200 nanometers.

71. The macroscopic scaffold of claim 65, wherein each of the plurality of peptides has a length of about 5 nanometers.
